(19) United States
(12) Patent Application Publication
(10) Pub. No.: US 2016/0354058 A1
(43) Pub. Date: Dec. 8, 2016

(54) MECHANICALLY DRIVEN ULTRASOUND SCANNING SYSTEM AND METHOD

(71) Applicant: SoniTrack Systems, Inc., Menlo Park, CA (US)

(72) Inventors: Jeffrey SCHLOSSER, Menlo Park, CA (US); Roman SOLEK, Pleasanton, CA (US)

(73) Assignee: SoniTrack Systems, Inc., Menlo Park, CA (US)

(21) Appl. No.: 15/173,479

(22) Filed: Jun. 3, 2016

Related U.S. Application Data
(63) Continuation of application No. PCT/US2014/068933, filed on Dec. 5, 2014.
(60) Provisional application No. 61/912,949, filed on Dec. 6, 2013.

(51) Int. Cl. A61B 8/14; A61B 8/00; A61B 8/08

(52) U.S. Cl. CPC A61B 8/145; A61B 8/483; A61B 8/4461; A61B 8/4405; A61B 8/4416; A61N 5/10

(57) ABSTRACT

Described herein are radiolucent imaging devices and methods capable of acquiring real-time 2D, 3D, or 4D images. By using remote-actuation techniques to mechanically drive an ultrasound transducer element in multiple directions, the majority of dense metallic components typically present in the ultrasound probe itself are eliminated. Therefore the system herein achieves both CT compatibility and radiation beam compatibility.

### CROSS-REFERENCE TO RELATED APPLICATIONS

This application is a continuation of International Application No. PCT/US2014/068933 filed Dec. 5, 2014, which claims the benefit of priority to U.S. Provisional Patent Application No. 61/912,949 filed Dec. 6, 2013, each of which is incorporated herein by reference in its entirety.

### FIELD OF THE INVENTION

The present invention relates to methods and apparatus for acquiring 3-dimensional (3D) or 4-dimensional (4D) ultrasound (US) images using a mechanically controlled imaging system. The present invention further relates to method and apparatus for acquiring ultrasound (US) images using a radiolucent US probe. More particularly, the present invention relates to the design of an US image system for use during radiation therapy or other medical therapies.

### BACKGROUND OF THE INVENTION

External Beam Radiation Therapy (EBRT) is used to treat more than half of all cancer patients worldwide. High dose conformality in EBRT is important for delivering curative dose to the target while sparing surrounding healthy structures, but anatomy motion poses a fundamental threat to realizing such conformity. The “holy grail” of EBRT is real-time imaging and tumor motion management during beam delivery without fiducial markers.

To provide real-time markerless imaging and tracking during beam delivery, real-time ultrasound (US) guidance systems have previously been developed and described: see “Telerobotic system concept for real-time soft-tissue imaging during radiotherapy beam delivery,” Med. Phys., 37 (12), December 2010, which is incorporated herein by reference in its entirety. However, integration of a traditional multi-element phased array ultrasound probe with real-time radiotherapy guidance systems has several important challenges including treatment beam interference, computed tomography (CT) imaging interference, workflow obstacles, and high cost.

The system herein describes a new radiolucent imaging device and method capable of acquiring real-time 3D (4D) US volumes and overcoming those challenges. By using remote-actuation techniques to mechanically drive a single ultrasound transducer element, in multiple directions, the majority of dense metallic components in the US probe itself are eliminated. Therefore the system herein achieves both CT compatibility and radiation beam compatibility. This advancement also overcomes major technical limitations of integrating a commercially-available 4D US probe with a real-time ultrasound guidance system for radiotherapy.

### SUMMARY OF THE INVENTION

By using remote-actuation techniques to mechanically drive one or more ultrasound transducer elements in multiple directions, the majority of dense metallic components typically present in an ultrasound probe are eliminated to provide for a system which achieves both CT compatibility and radiation beam compatibility.

One embodiment of a system for 3D or 4D imaging may generally comprise a single imaging element configured to result in a nominal or minimal imaging artifact (e.g., such that any resulting imaging artifact, if present, does not interfere with the imaged region) and minimal radiation absorption when irradiated (e.g. for minimizing dose interference with radiotherapy), one or more actuators which can be positioned remotely from the at least one imaging element, and a mechanism coupling the single imaging element with the one or more actuators to drive the at least one imaging element in at least two degrees of freedom in order to acquire 3D imaging information, wherein the mechanism is configured to result in a nominal imaging artifact and minimal radiation absorption when irradiated. For the components which present a nominal or minimal imaging artifact, such components may be effectively radiolucent which generally results in a nominal or minimal imaging artifact (e.g., such that resulting imaging artifacts, if present, do not substantially interfere with the imaged region) and minimal interference with radiotherapy delivery (e.g. for minimizing radiotherapy discrepancies between planned dose and delivered doses which may occur when relatively dense materials are used, as further described herein).

Yet another embodiment of a system for imaging may generally comprise at least one imaging element configured to result in a nominal imaging artifact when irradiated, one or more mechanical actuators located remotely from an imaging field of the at least one imaging element, and a mechanical coupling of the at least one imaging element with the one or more actuators which are remotely located via an extended transmission to drive the at least one imaging element in at least one degree of freedom in order to acquire imaging information, wherein a first portion of the mechanical coupling attached to the at least one imaging element extends within an irradiating field and a second portion of the mechanical coupling attached to the one or more mechanical actuators extends outside of the irradiating field.

Yet another embodiment for a system for imaging may generally comprise at least one imaging element configured to result in a nominal imaging artifact when irradiated, one or more radiolucent mechanical actuators, and a coupling between the at least one imaging element with the one or more radiolucent mechanical actuators to drive the at least one imaging element in at least one degree of freedom in order to acquire image information.

Yet another embodiment for a system for imaging may generally comprise at least one imaging element configured to result in a nominal imaging artifact when irradiated, one or more radiolucent mechanical actuators, and a coupling between the at least one imaging element with the one or more radiolucent mechanical actuators to drive the at least one imaging element in at least one degree of freedom in order to acquire image information.

Yet another embodiment for a system for imaging may generally comprise providing at least one imaging element, one or more actuators, and a mechanism to couple the at least one imaging element with the one or more actuators to drive the at least one imaging elements in at least two degrees of freedom in order to acquire 3D or 4D image information, and scanning a region of a body via the at least one imaging element while maintaining a radiolucent profile of at least one imaging element.

Yet another embodiment for a system for imaging may generally comprise providing at least one imaging element, one or more mechanical actuators located away from an imaging field, and a mechanical coupling of the at least one imaging element with the remotely-located one or more mechanical actuators via extended transmissions to drive the at least one imaging elements in at least one degree of freedom in order to acquire image information, and scanning a region of a body via the imaging element while maintaining a radiolucent profile of at least one imaging element.

Yet another embodiment for a system for imaging may generally comprise providing at least one imaging element, one or more actuators, and a mechanism to couple the at least one imaging element with the one or more actuators to drive the at least one imaging elements in at least two degrees of freedom in order to acquire image information, and scanning a region of a body via the at least one imaging element while maintaining a radiolucent profile of the at least one imaging element, wherein the imaging element is moved relative to the region of the body in a pattern formed by a series of data collection planes which collectively forms an image.

### BRIEF DESCRIPTION OF THE DRAWINGS

FIGS. 1A and 1B illustrate CT scans of a phantom (FIG. 1A) without and (FIG. 1B) with a commercially available probe, e.g., Philips X6-1 4D US probe (Koninklijke Philips N. V., Netherlands) imaging underlying targets. Severe metal artifacts in (FIG. 1B) preclude simultaneous US/CT imaging for radiotherapy planning.

FIG. 1C illustrates an example of a commercially available US machine, e.g., Philips iU-22 US machine (Koninklijke Philips N. V., Netherlands) form factor.

FIGS. 2A to 2D illustrate CT scans of metallic elements contained in one variation of the MDUSS design. Notice the absence of significant metal artifacts. FIGS. 2A and 2B show a single 17 mm US transducer element with a 2D slice view for illustrative purposes and FIGS. 2C and 2D show a 1.5 mm metallic (steel) cable coil and a 2D slice view also shown for illustrative purposes.

FIGS. 3A and 3B illustrate one embodiment of the MDUSS device. FIG. 3A shows a mechanical design embodiment and the detailed insert in FIG. 3A details operation of the peg-in-slot mechanism. FIG. 3B illustrates one possible data collection pattern for this embodiment.

FIGS. 4A and 4B illustrate another possible embodiment of the MDUSS device. FIG. 4A shows a mechanical design embodiment. FIG. 4B illustrates one possible data collection pattern for this embodiment.

FIGS. 5A to 5C illustrates another possible embodiment of the MDUSS device. FIG. 5A illustrates a mechanical design embodiment. FIG. 5B illustrate one possible data collection pattern for this embodiment. FIG. 5C illustrates another possible data collection pattern for this embodiment.

FIG. 6 shows a 3D rendering of another possible embodiment of the MDUSS device.

FIGS. 7A and 7B detail the design embodiment shown in FIG. 6. FIG. 7B shows a 2D cross section in the vicinity of the ultrasound imaging element.

FIGS. 8A and 8B depict one aspect of the motion mechanism of the design embodiment in FIG. 6 and FIG. 7.

FIG. 9 illustrates a reconstructed 3D ultrasound volume collected with the embodiment shown in FIG. 3. The volume is depicted with three orthogonal 2D slices.

### DETAILED DESCRIPTION OF THE INVENTION

Use of existing 4D US probes for real-time radiation therapy guidance presents several important technical challenges including treatment beam interference, computed tomography (CT) imaging interference, workflow obstacles, and high cost. The following several paragraphs detail each challenge.

Treatment beam interference. Presence of a traditional US probe in the treatment field poses a challenge for radiation therapy planning. Radiation beams can pass through the US probe, causing dose deviations that negatively affect patient outcomes. This problem can be addressed using two approaches: (1) plan treatment beam directions to avoid US probe interference; (2) deliver radiation directly through the US probe and incorporate the hardware's perturbing effect into the dose calculation process. However, both approaches have substantial limitations. In the first approach, the US probe can preclude use of certain important beam angles and result in sub-optimal dose sculpting around the target, especially for patients with superficial tumors. Furthermore, careful selection of a non-interfering beam set adds time to the treatment planning process. In the second approach, significant dose uncertainties resulting from beam delivery through high-density US probes can negatively impact the actual dose deposited at the target and surrounding organs at risk. Existing 4D probes collect volumes via 2D matrix arrays or wobbling 2D arrays, both of which have high metal content due to the large number of active phased-array elements, complex on-board electronics processing, and/or on-board wobbler motors.

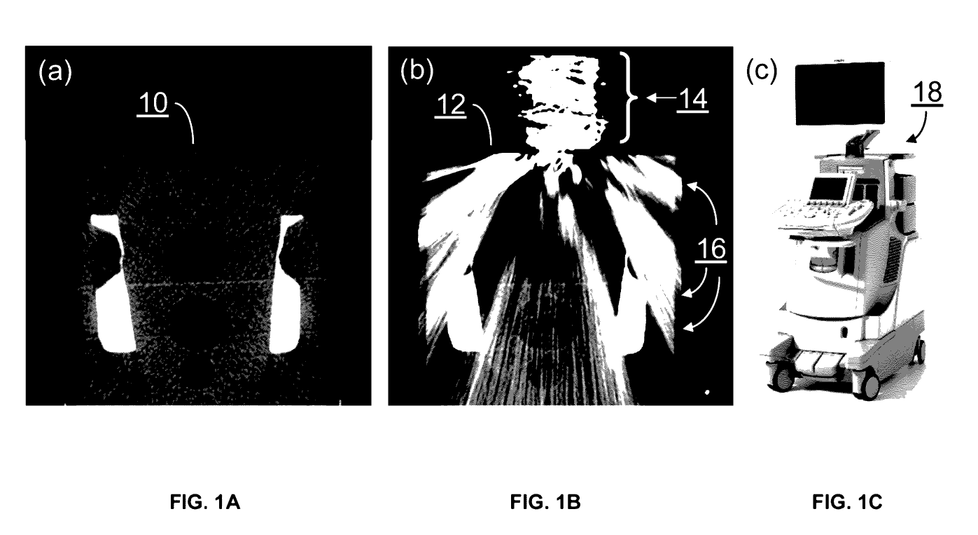

CT imaging interference. During the planning phase of radiotherapy, patients receive a CT scan that is used to simulate radiation propagation through the patient's body. For real-time US guided radiotherapy, an US image is collected with the CT scan and registered to the CT. During radiation delivery, US volumes are processed with respect to the fused CT/US set and used to determine real-time 3D tumor position with respect to the LINAC. However, metallic components within existing 4D probes preclude simultaneous US/CT imaging. FIGS. 1A to 1B display the results of a CT scan without 10 and with 12 the presence of a commercially available Philips X6-1 4D US transducer 14 (Koninklijke Philips N. V., Netherlands), indicating severe metal artifacts 16 caused by the 4D probe 14. For this reason, with existing probes, CT and US images can be collected non-synchronously. Any patient anatomy movement between collection of CT and US images negatively impacts the accuracy of tumor position estimates computed by the guidance system at the time of radiotherapy beam delivery. In addition, since US probe pressure causes anatomy deformation, lack of probe pressure during CT acquisition can cause differentials in patient anatomy between planning and treatment phases, negatively impacting the actual dose delivered at the time of treatment. Note that magnetic resonance (MR) images can he used instead of CT images for radiotherapy planning. Existing US probes also cause MR interference due to high metal content, and thus the above discussion applies to MR planning images as well.

Workflow obstacles. It is desirable that new image guidance technologies introduced into the clinic are minimally cumbersome to facilitate adoption. Processing units for existing 4D US systems are generally quite large as shown in FIG. 1C, 18, and probe cable lengths are typically limited to approximately 5 feet. These limitations become a burden for image-guided radiotherapy, since the US processing unit must be carefully placed in a particular position and orientation that avoids collisions with the LINAC while maintaining enough slack in the probe cable to enable constraint-free patient imaging and avoid pulling the cable taut.

Cost. Long-term exposure to direct and scatter radiation can eventually degrade US probe performance. The US probe may therefore need to be periodically replaced when used to monitor anatomy during radiation therapy. Existing phased-array transducers are expensive, and periodically replacing them may be cost-prohibitive. Even without periodic probe replacement, the up-front cost of an image guidance system with existing phased-array probes could be high.

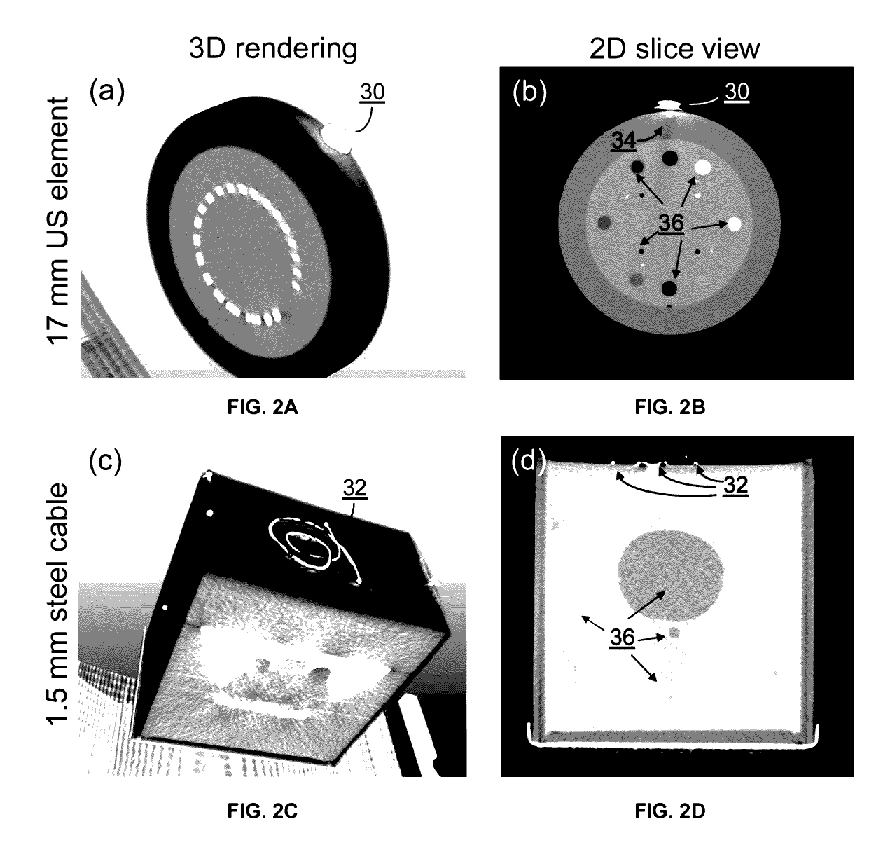

The system herein describes a novel Mechanically Driven UltraSound Scanning (MDUSS) system and method that overcomes the obstacles listed in the paragraphs above. A first feature is using mechanical means to steer a US transducer element in multiple directions to acquire 4D (real-time 3D) US volumes. A second feature is using remotely-located actuators and sensors (or actuators/sensors that lack metal content such as pneumatic or hydraulic actuators or fiber optic sensors) to steer the single transducer element, thus enabling the elimination of the majority of the metal contained in the US probe itself. Using these principles, the primary sources of metal in the probe can be the small amount of metal in a US transducer element and the electrical cable that transmits signals from the element to an external processing system. FIGS. 2A and 2B show a respective 3D rendering and a corresponding 2D slice view which demonstrate that a single US transducer element 30, e.g., 17 mm diameter transducer element (or similar), and as shown in FIGS. 2C and 2D which show a respective 3D rendering and a corresponding 2D slice view of a steel cable 32, e.g., 1.5 mm diameter cable (or similar), do not contain large enough volumes of metal to cause significant CT artifacts 34 or to impair the ability to visualize underlying structures 36 in the CT scan. Therefore, the system herein enables simultaneous CT/US imaging during the radiotherapy planning phase which minimizes the chances of CT/US mis-registration due to anatomy movement.

An additional benefit of using MDUSS to minimizing metallic probe components is that radiation beams can be sent directly through the MDUSS device, enabling physicians to maximize the number of available beam directions and thus maximizing chances to achieve desired dose distributions at the target and surrounding healthy organs. Another additional benefit of the MDUSS device is that the amount of data processing and electrical power required to control a single (or small number of) US transducer element is significantly less than the processing and power required for a dense phased array. Therefore the single-element MDUSS device can be controlled using a USB connection to a laptop, smart phone, tablet, or other small processing unit, which minimizes form factor, minimizes cost, and maximizes portability.

The following sections describe several possible preferred embodiments of the MDUSS device using remotely located actuators and sensors. Note that other embodiments are also possible, including those where the actuators and sensors are not located remotely but instead are made using non-metallic (or otherwise radiolucent) materials, those where different combinations of motors and sensors are used, those where motion of the transducer element is designated about different axes, those where different data collection patterns are used, and those where more than one transducer element is used.

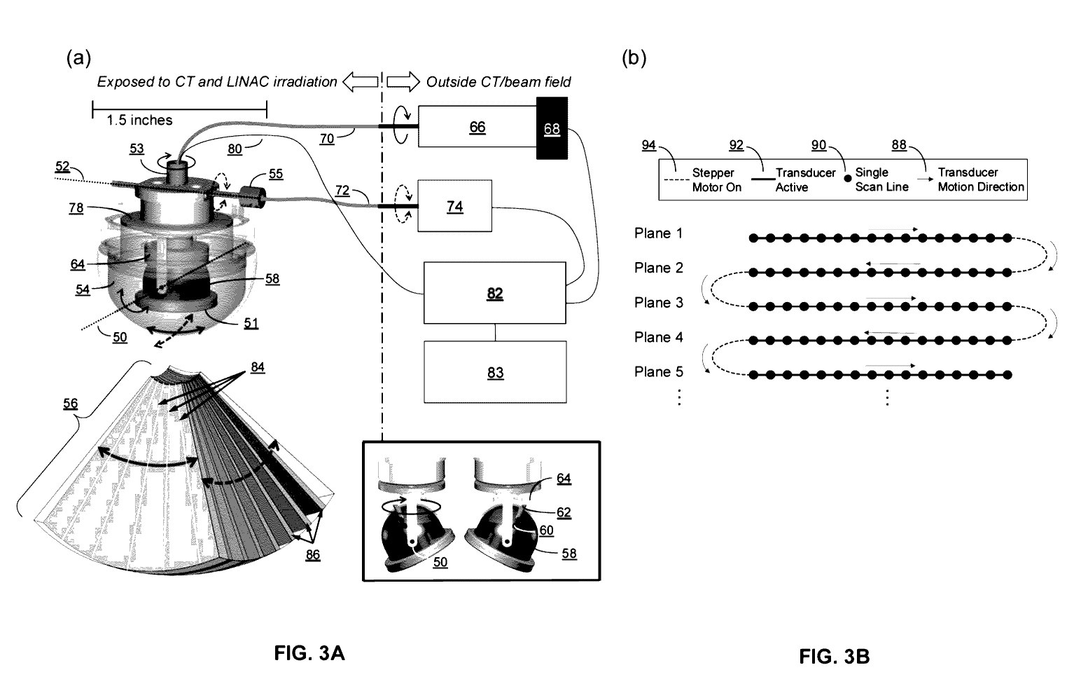

FIG. 3A illustrates a perspective assembly view of the basic elements of one design embodiment of the MDUSS device. A single transducer element 51 (e.g., 17-mm diameter or similar) rotates about axis 1 50 and axis 2 52 within a fixed, fluid-filled chamber 54 in order to produce a 3D volume 56 of US information. Rotation about axis 1 50 (solid arrows, FIG. 3A) is achieved using, e.g., a motion ball 58 that is physically coupled to the transducer element 51. A slot 60 defined along the top of the motion ball 58 houses a peg 62 that is non-concentrically coupled to a rotating disk 64. Constant speed disk 64 rotation causes the peg 62 to translate within the slot 60, resulting in a sinusoidal-like rotation of the motion ball 58 about axis 1 50 (insert, FIG. 3A). Constant speed rotation of the disk 64 is achieved using, e.g., a remotely-located DC motor 66, and encoder 68 combination coupled to the disk 64 via a thin, torsionally-stiff flex shaft 70. Rotation of the assembly about axis 2 52 (dotted arrows, FIG. 3A) may be achieved by coupling a second flex shaft 72 directly to axis 2 52 at one end, and directly to the output of a stepper motor 74 at the other end. A flexible seal 78 between the assembly and the fixed fluid-filled chamber can prevent fluid leakage. A shielded cable 80 conducts a voltage signal from the transducer element 51. One or more remotely-located processing boards 82 send a receive signals from the transducer element 51, motors 66, 74, and sensors 68. The processing boards can contain signal amplifiers and conditioning circuits necessary for transmitting and receiving US transducer element signals. A computer 83 can coordinate signals received and sent from the processing board(s).

The probe design as shown and described may include alternative designs as well. For instance, the US transducer element could be replaced with an optical imaging element, photoacoustic imaging element, camera, or other imaging element (with minimum metal content) to enable collection of other types of medical images using the same principles described above. Furthermore, the system can be used as part of a robotic ultrasound image guidance system for radiotherapy, a guidance system for radiotherapy without a robotic holding device, or in applications outside radiation therapy. These variations may be applicable not only to the embodiment described above but are also intended to be applicable to each of the other embodiments as described herein.

The majority of parts to the left (for discussion purposes) of the vertical dotted line in FIG. 3A (areas exposed to CT and LINAC radiation) can be fabricated using plastic (or other radiolucent material) which remains radio-lucent. The transducer element 51 (e.g., having a diameter of 17 mm or similar) and the flex shafts 70, 72 (e.g., having a 1.5 mm diameter cable or similar) could contain relatively small volumes of metal, but these quantities do not have significant effect on CT image quality or on radiation delivery through the hardware (see FIG. 2). Components located to the right (for discussion purposes) of the dotted line in FIG. 3A such as motors, encoders, processing boards, and control boards contain large quantities of metal, but are mounted arbitrarily and remotely from the CT imaging field and prospective path of radiation beams.

FIG. 3B schematically illustrates a possible US data collection pattern for the MDUSS embodiment shown in FIG. 3A. The motions of the stepper and DC motors are preferably coordinated via a computer 83 to steer the US transducer element 51 appropriately to collect 3D US data. The arrows 88 and lines 92, 94 in FIG. 3B depict the motion path of the transducer. Constant sinusoidal-like motion of the transducer element 51 enables the rapid collection of a series of scan lines 84 (represented schematically by dots 90 in FIG. 3B) that together form a single planar 2D US image (solid lines 90 in FIG. 3B). The duty of the stepper motor 74 can be to periodically redirect the transducer element 51 such that it moves to collect a new US plane in a slightly different spatial location than the last. Dotted lines 94 in FIG. 3B indicates periods of time when the stepper motor is active and the transducer element is not collecting image data. Short bursts from the stepper motor 74—commanded near the inflection points of the sinusoidal-like motion produced by the DC motor 66—rotate the transducer element 51 slightly about axis 2 52 and result in a new data collection plane 86. In this way, multiple 2D slices 86 rotated about an axis 52 can be collected, compromising a 3D volume 56. In this example, once a first plane of images are collected in a first direction, the rotation of the transducer element 51 about axis 2 52 may then allow for the collection of images in a second plane adjacent to the first plane which are collected in a second direction opposite to the first direction. Additional images in a third plane may then be collected in the first direction in a similar manner, and so forth until a desired number of data collection planes 86 have been imaged.

The US data collection pattern shown in FIG. 3B is intended to be exemplary of one possible pattern. However, this is not intended to be limiting as any number of other various other data collection patterns (as shown below and as also known in the art) may be applicable to this embodiment as well as any of the other embodiments described, as practicable.

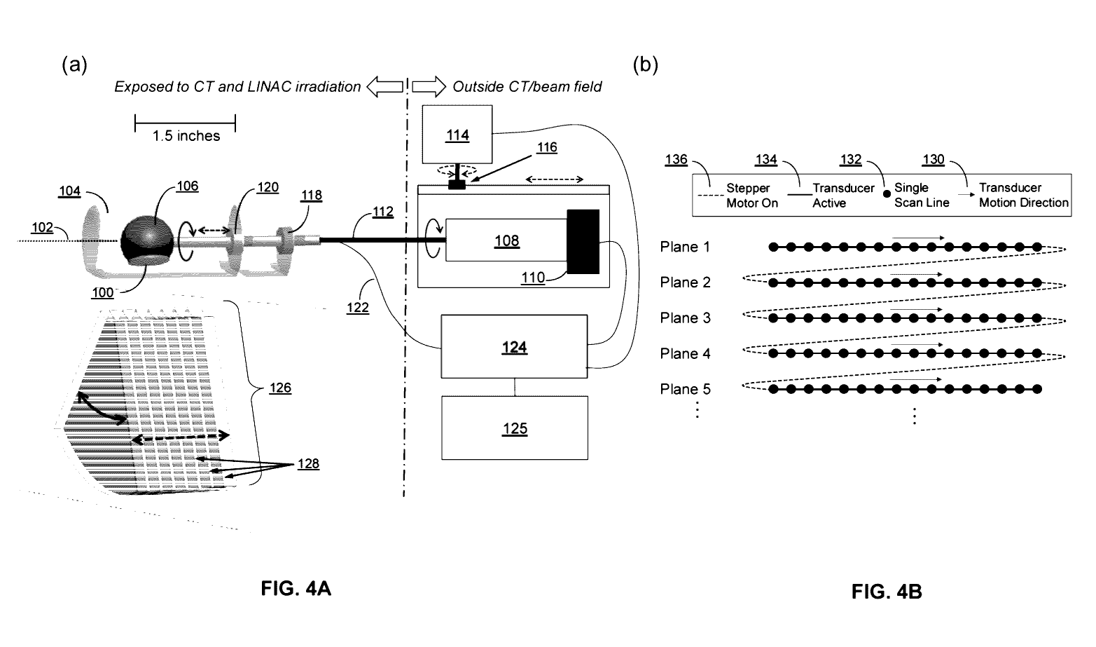

FIG. 4A shows another embodiment of the MDUSS device. In this design, the transducer element 100 rotates and translates about a single axis 102 within a fixed, fluid filled chamber 104. The transducer 100 is affixed to a spherical part 106 to minimize resistance while rotating within the fluid. Constant speed rotation about the axis 102 (solid arrows, FIG. 4A) is achieved using, e.g., a DC motor 108 and encoder 110 directly coupled to the sphere 106 via a rigid shaft 112. The shaft can be supported using one or more bearings or bushings 118. Translation of the shaft 112 (dotted arrows, FIG. 4A) can be achieved using, e.g., a fixed stepper motor 114 that drives the mobile DC motor 108 unit using a rack and pinion system 116. In other variations, other types of actuation mechanisms may be utilized to drive the motion of the shaft. A fluid seal 120 between the rotating/translating shaft 112 and the fixed fluid chamber 104 prevents fluid leakage. A shielded cable 122 conducts a voltage signal from the transducer element 100. One or more remotely-located processing boards 124 send receive signals from the transducer element 100, motors 108, 114, and sensors 110. A computer 125 can coordinate signals received and sent from the processing board(s). All components to the left of the dotted line (FIG. 4A) other than the transducer element 100 can be fabricated out of non-metallic (or otherwise radiolucent) materials, including the rigid shaft, as these components are exposed to CT and LINAC irradiation while the components shown to the right of the dotted line may be remotely located so as to be outside the CT and LINAC field. The components such as the transducer element 100, shaft 112, shielded cable 122 may be fabricated from metallic materials and/or include metallic components but their respective diameters may be relatively small enough, as described above, so as to minimize any CT artifacts which do not impair the ability to visualize underlying structures in the CT scan.

FIG. 4B schematically illustrates a possible US data collection pattern for the MDUSS embodiment shown in FIG. 4A. The motions of the stepper and DC motors may be coordinated by a computer 125 to steer the US transducer element 100 appropriately to collect. 3D US data. The arrows 130 and lines 134, 136 in FIG. 4B depict one example for the motion path of the transducer. Constant sinusoidal-like motion of the transducer element 100 enables the rapid collection of a series of scan lines (represented schematically by dots 132 in FIG. 4B) that together form a single planar 2D US image (solid lines 134 in FIG. 4B). The duty of the stepper motor 114 can be to periodically redirect the transducer element 100 such that it moves to collect a new US plane in a slightly different spatial location than the last. Dotted lines 136 in FIG. 4B indicate periods of time when the stepper motor is active and the transducer element is not collecting image data. As the transducer 100 rotates away from the imaging field, the stepper motor 114 translates the transducer in the direction of the next parallel imaging plane 128 before the transducer finishes its continuous 360 degree rotation back towards the imaging area. In this way, multiple 2D slices 128 translated about an axis 102 can be collected, compromising a 3D volume 126.

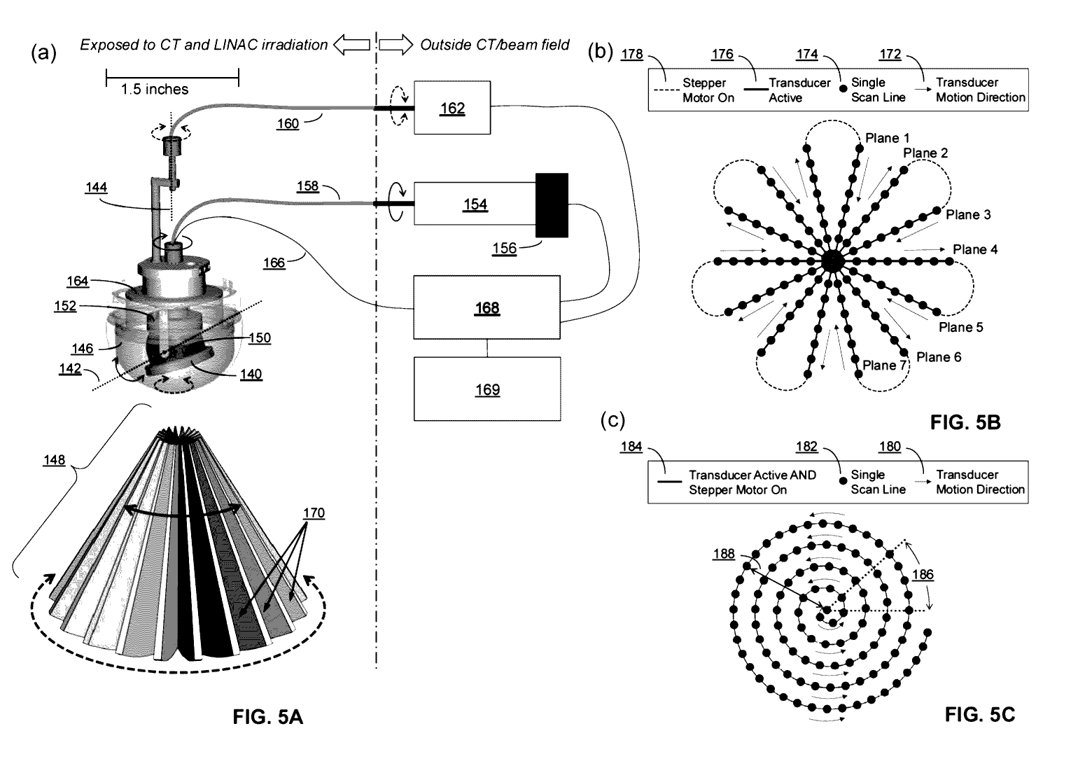

FIG. 5A shows another embodiment of the MDUSS device. This design is similar to the design in FIG. 3, with the major exception being the orientation of the second rotation axis 144. A single transducer element 140 rotates about axis 1 142 and axis 2 144 within a fixed, fluid-filled chamber 146 in order to produce a 3D volume 148 of US information. Rotation about axis 1 142 (solid arrows, FIG. 3) is achieved using a similar motion ball 150 and disk 152 setup as the design in FIG. 3 (see insert, FIG. 3A). Rotation of the disk 152 is achieved using a remotely-located DC motor 154 and encoder 156 combination coupled to the disk 152 via a thin, torsionally-stiff flex shaft 158. Rotation of the assembly about axis 2 144 (dotted arrows, FIG. 3) is achieved by coupling a second flex shaft 160 directly to axis 2 144 at one end, and directly to the output of a stepper motor 162 at the other end. A torsionally-flexible seal 164 between the assembly and the fixed fluid-filled chamber prevents fluid leakage. A shielded cable 166 conducts a voltage signal from the transducer element 140. One or more remotely-located processing boards 168 send a receive signals from the transducer element 140, motors 154, 162, and sensors 156. A computer 169 can coordinate signals received and sent from the processing board(s). As previously described, all components to the left of the dotted line (FIG. 5A) other than the transducer element 140, shaft 158, and second flex shaft 160 can be fabricated out of non-metallic (or otherwise radiolucent) materials as these components are exposed to CT and LINAC irradiation while the components shown to the right of the dotted line may be remotely located so as to be outside the CT and LINAC field.

FIG. 5B schematically illustrates a possible US data collection pattern for the MDUSS embodiment shown in FIG. 5B. The motions of the stepper and DC motors are preferably coordinated by a computer 169 to steer the US transducer element 140 appropriately to collect 3D US data. The arrows 172 and lines 176, 178 in FIG. 5B depict the motion path of the transducer. Constant sinusoidal-like motion of the transducer element 140 enables the rapid collection of a series of scan lines (represented schematically by dots 174 in FIG. 5B) that together form a single planar 2D US image (solid lines 176 in FIG. 5B). The duty of the stepper motor 162 can be to periodically redirect the transducer element 100 such that it moves to collect a new US plane in a slightly different spatial location than the last. Dotted lines 178 in FIG. 5B indicate periods of time when the stepper motor is active and the transducer element is not collecting image data. Short bursts from the stepper motor 162—commanded near the inflection points of the sinusoidal-like motion produced by the DC motor 154—rotate the transducer element 140 slightly about axis 2 144 and result in a new rotated data collection plane 170. In this way, multiple 2D slices 170 rotated about a central axis 144 can be collected such that the one or more data collection planes are configured in a radial pattern about a central axis, compromising a 3D volume 148.

FIG. 5C schematically illustrates another possible US data collection pattern for the MDUSS embodiment shown in FIG. 5A. Note that the US volume 148 shown in FIG. 5A applies to the data collection pattern in FIG. 5A, not the pattern in FIG. 5C. The arrows 180 and line 184 in FIG. 5C depict a motion path of the transducer. Unlike the data collection pattern in FIG. 5B, a series of 2D slices do not compromise a 3D volume. Instead, the transducer is steered in a spiral motion trajectory about a center or central axis by continuously controlling the motion of the stepper motor 162 and DC motor 154. The stepper motor determines the angular position 186 of the transducer (or scan line 182) with respect to the spiral, and the DC motor determines how far away from the center of the spiral the transducer is pointing 188. Therefore to collect scan lines 182 that are evenly spaced in the time domain, the stepper motor and the DC motor can move at variable rates. Towards the center of the spiral, the DC motor and stepper motor must move quickly because relatively few scan lines are collected within a certain angular transducer displacement 186 and within a certain distance displacement flow the spiral center 188. Towards the outside edges of the spiral, the DC motor and stepper motor slow since many more scan lines are collected within the same angular transducer displacement and distance displacement from the center.

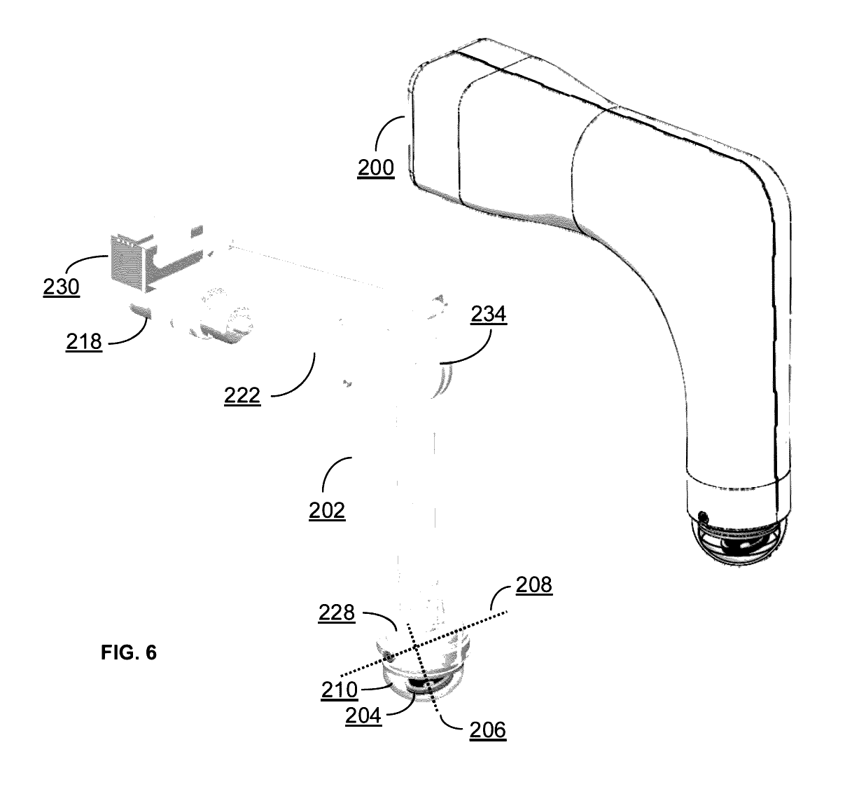

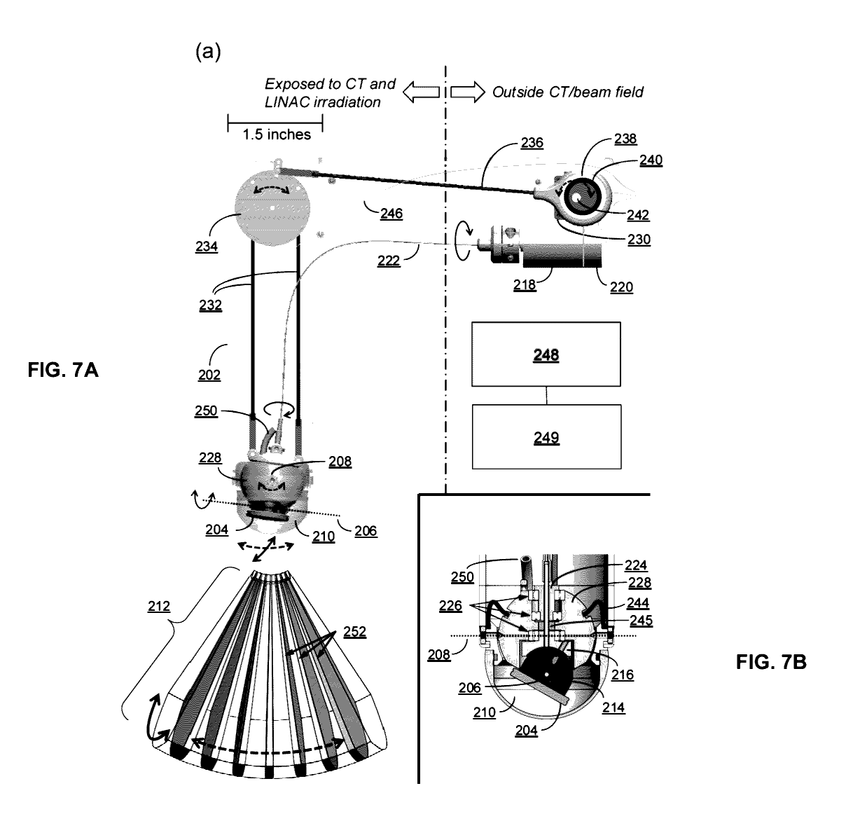

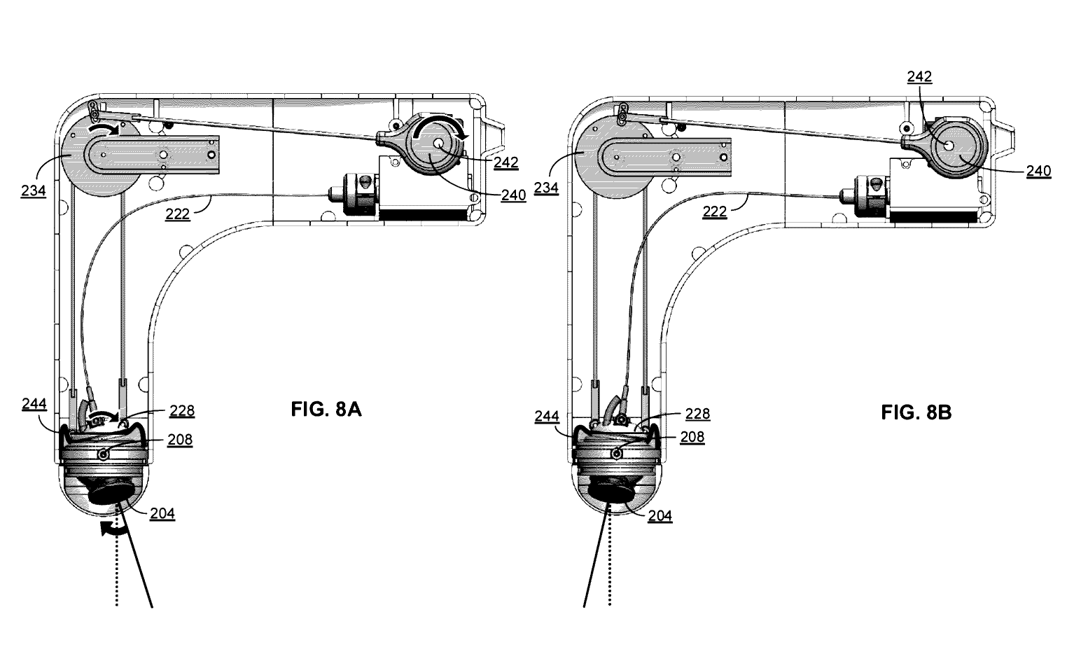

FIGS. 6, 7, and 8 show another embodiment of the MDUSS device in an opaque casing 200 and alternatively in a transparent casing 202. FIG. 6 illustrates perspective views while FIGS. 7A and 8A-8B show exemplary side views. This embodiment is similar to the basic design in FIG. 3, with the major exception being that a rigid linkage system connects axis 2 208 to the stepper motor 230 instead of a flexible shaft as in 72, FIG. 3A. Referring to FIG. 6-8, a single transducer element 204 rotates about axis 1 206 and axis 2 208 within a fixed, fluid-filled chamber 210 in order to produce a 3D volume 212 of US information. Rotation about axis 1 206 (solid arrows, FIG. 7) produces individual 2D US planes 252 and is achieved using a similar motion ball 214 and disk 216 setup as the design in FIG. 3. Rotation of the disk 216 is achieved using a remotely-located DC motor 218 and encoder 220 combination coupled to the disk 216 via a thin, torsionally-stiff flex shaft 222. In this embodiment, the disk 216 is coupled to the flex shaft 222 using a rigid shaft assembly 224 supported by a set of plastic bearings 226 inside a plastic pivot sphere 228. Rotation of the assembly about axis 2 208 (dotted arrows, FIG. 7) is achieved by coupling the pivot sphere 228 to a stepper motor 230 via a linkage system. The linkage system consists of a parallel set of rods 232 transmitting 1:1 motion between the pivot sphere 228 and a linkage disk 234. A second rod 236 couples the linkage disk 234 to a bearing 238 that supports rotation of a non-concentric 240 disk rigidly coupled to the stepper motor shaft 242. The result is a system that yields an arbitrarily large stepper motor torque reduction as a function of the non-concentric disk offset. A flexible seal 244 between the assembly and the fixed fluid-filled chamber and a second seal 245 between the pivot sphere 228 and shaft assembly 224 prevent fluid leakage. A sealable tube 250 penetrating through the pivot sphere 228 allows the fluid chamber 210 to be filled. A shielded cable 246 conducts a voltage signal from the transducer element 204. One or more remotely-located processing boards 248 send a receive signals from the transducer element 204, motors 218, 230, and sensors 220. A computer 249 can coordinate signals received and sent from the processing board(s).

FIGS. 8A and 8B illustrates an example of motion of the linkage system to achieve rotation of the pivot sphere 228 and transducer element 204 about axis 2 208. Motion of the linkage system is activated by rotation of the stepper motor and non-concentric disk 240 about the stepper motor shaft 242. Non-concentric disk 240 may also be a disk which is pivoted or rotated about a shaft 242 which is non-centrally located relative to the disk such that rotation of the disk about the shaft 242 results in an eccentric rotational motion of the disk. Bending of the flexible shaft 222 as the pivot sphere 228 rotates is apparent in FIGS. 8A and 8B. Stretching and bending of the flexible seal 244 as the pivot sphere 228 rotates is also apparent in the figures.

The US data collection pattern for the MDUSS embodiment shown in FIGS. 6-8 can be the same as the pattern shown in FIG. 3B. The motions of the stepper and DC motors are preferably coordinated via a computer 249 to steer the US transducer element 204 appropriately to collect 3D US data. In FIG. 7A, multiple 2D slices 252 rotated about an axis 208 can be collected, compromising a 3D volume 212.

The majority of parts to the left of the vertical dotted line in FIG. 7A (areas exposed to CT and LINAC radiation) can be fabricated using plastic (or other radiolucent material). The parallel rods 232 and second rod 236 can be fabricated out of carbon fiber. The transducer element 204, and flex shaft 222, and parts of the rigid shaft assembly 224 could contain small volumes of metal, but these quantities do not have significant effect on CT image quality or on radiation delivery through the hardware (see FIG. 2). Components located to the right of the dotted line in FIG. 7 contain large quantities of metal, but are mounted remotely away from the CT imaging field and prospective path of radiation beams.

In the embodiment shown in FIGS. 6-8, motor 218, 230 stators may be rigidly fixed in space with respect to the fluid casing 210. Normally the motor rotors are commanded to rotate with respect to the motor stators to produce motion of the transducer element 204 as described above. If motor stators rotate with respect to the fluid casing 210, the transducer element 204 could rotate with respect to the fluid casing without relative motion between the motor stator and rotors. Such motion of the transducer element 204 is generally unwanted and difficult to model, and thus the motor stators and fluid casing are rigidly connected via the probe casing 202 in this design although alternative variations are possible.

Another unique feature of the embodiment in FIGS. 6-8 is that the parts of the embodiment exposed to LINAC irradiation are axially symmetric. If LINAC radiation is delivered through a probe at a fixed beam angle while said probe is undergoing mechanical motion, the absorption profile of the beam through said probe could change, thus perturbing the dose delivered to the patient. One way to account for this perturbation is to capture the probe in the patient's planning scan while the probe undergoing mechanical motion. In this way, the “average” absorption profile through the probe can be computed using a motion-blurred planning scan. Since the mechanical motion is predictable and reproducible during treatment, the “blurred” planning scan can be used to compute “average” dose delivered to the patient. Another technique to minimize mechanical motion dose discrepancies is to design the mechanical system such that mechanical motion does not cause significant changes in absorption profile. One way to achieve this is to design the parts with significant material volume to be axially symmetric about their rotation axis to the greatest extent possible. In the embodiments in FIGS. 6-8, the parts undergoing mechanical motion that have the largest material volumes exposed to radiation are the pivot sphere 228 and linkage disk 234. Therefore the pivot sphere 228 is designed to be mostly axially symmetric with respect to its rotation axis 208, and similarly the linkage disk 234 is mostly axially symmetric with respect to its own rotation axis.

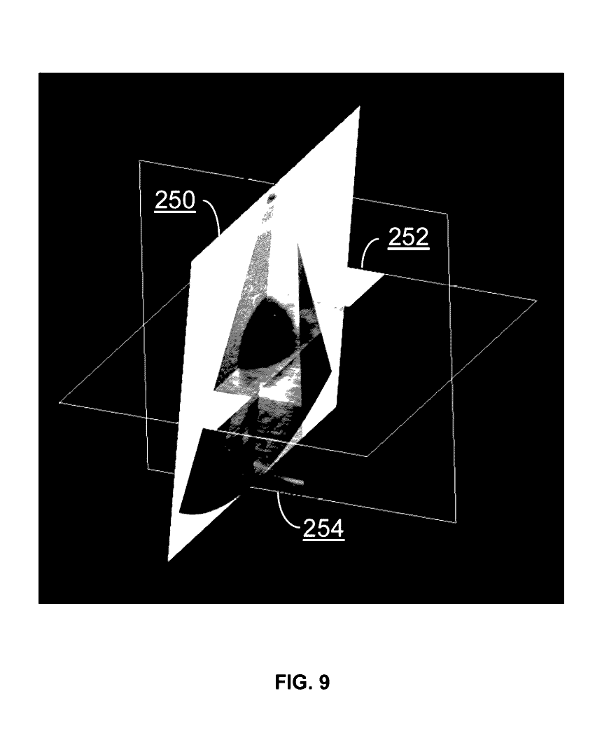

Once a series of US scan lines is collected according to FIG. 3B, FIG. 4B, FIG. 5B, FIG. 5B, or other possible embodiments, they may be compiled into a continuous 3D volume for purposes of 4D imaging. The spatial location and orientation of each scan line is determined using a geometrical model of the probe in combination with the known positions of the stepper motor and DC motor at the time each scan line is collected. Standard data binning methods can be used to interpolate data in the space between scan lines and reconstruct 3D volumes. As an intermediate step, in the case of data collection methods shown in FIGS. 3B, 4B, and 5B, scan lines can first be compiled into a set of distinct 2D US images, and then the 2D US images can be interpolated into a full 3D volume. FIG. 9 shows an example of three orthogonal slices 250, 252, 254 through a reconstructed 3D volume collected with the design embodiment shown in FIG. 3.

By eliminating one of the degrees of freedom (DOFs) in the embodiments shown in FIGS. 3-8 (or similar embodiments), a simplified radiolucent, mechanically actuated 2D US imaging probe can be achieved. For example, in FIG. 3A, if axis 2 52, flex shaft 72, and the stepper motor 74 are removed, the transducer element 51 can only be mechanically steered in one DOF. However, with this a simpler design embodiment is achieved that collects real-time 2D US images in a radiolucent package.

While the previously described design embodiments (FIGS. 3-8 and related embodiments) have involved the use of a single US transducer element, note that MDUSS principles also apply to steering multiple US transducer elements. For example, the single large US transducer element could be replaced by an array of smaller US elements rigidly fixed together and mechanically steered in one or more DOFs using MDUSS principles. Each array element, could have a miniature electrical cord extending to remotely located US processing board(s) (e.g. 82 in FIG. 3A) forming a cord bundle. As long as the total volume of the electrical cord bundle does not exceed a certain threshold causing metal CT artifacts and high radiotherapy dose absorption, a multi-transducer element MDUSS design embodiment can still be sufficiently radiolucent. Alternatively, a small electronics processing board located within the radiation field could process and condense multiple transducer element signals into a lesser set of signal cables extending outside the beam field. Again, as long as the volumetric metal content of the on-board electronics is sufficiently small, such as design could be radiolucent for purposes of radiotherapy. A multi-transducer element MDUSS embodiment could utilize phased-array imaging techniques to achieve dynamic depth focusing and other advanced US imaging features. A multi-transducer element MDUSS embodiment could achieve 3D or 4D US imaging by electronically controlling image acquisition in one plane, and controlling out-of-plane transducer element motion in a single mechanical DOF using a remotely located or radiolucent actuator.

Previously described design embodiments (FIGS. 3-8 and related embodiments) have involved the use of remotely located actuators and sensors to drive motion of the transducer element while maintaining a radiolucent probe profile. Note that a radiolucent probe profile can also be achieved by employing onboard actuators and sensors that are themselves radiolucent. In other words, actuators and sensors that drive the motion of the transducer element can be located within areas exposed to LINAC and CT radiation if they are sufficiently radiolucent. For example, in FIG. 3A, radiolucent actuators (such as hydraulic or pneumatic actuators) can be directly connected to the distal end connectors 53, 55 of the flex shafts in order to drive motion about axis 1 50 and axis 2 52 without the need for long flexible shafts 70, 72. Similarly, to sense motion about axis 1 and axis 2, radiolucent sensors (such as fiber optic sensors) can be mounted directly adjacent to the radiolucent actuators.

The MDUSS technology described herein has potential to impact medical imaging outside the field of radiation therapy. A few specific applications are described below.

2D US imaging requires training and expertise in order to localize and visualize target anatomy, since 2D US imaging is highly sensitive to small changes in probe orientation. The field of view of 4D US is much larger, decreasing operator variability and enabling less-experienced users to obtain volumetric images that can be analyzed on-site or by a remote physician. The MDUSS technology described herein can be packaged into a lightweight, portable, low-cost 4D US probe suitable for deployment in battle, field expeditions, developing countries, space missions, and other areas outside the clinic. For these applications, since radiolucency is not a design requirement, non-radiolucent actuators (e.g. motors) controlling the US transducer degrees of freedom can be mounted close to the transducer itself, eliminating the need for lengthy transmission systems shown in FIGS. 3-8 (or similar embodiments).

Previous US transducers are not generally capable of integration with other modalities because of their high metal content. However, MDUSS with remotely located actuators (or radiolucent actuators) offers real-time soft-tissue imaging with very low probe metal content that can be synchronously acquired with PET, CT, and MR images to enhance utility of each respective modality. For example, MDUSS can be synchronously acquired with CT, MRI, or PET to provide an accurate breath-gating signal based on real-time measurements of target anatomy displacements, decreasing motion-blur artifacts that plague these modalities during abdominal imaging. As another example, US flow/strain imaging, molecular microbubble imaging, and other unique US capabilities can be fused with PET, CT, or MR images to add functional information to aid in diagnostic imaging or image-guided interventions, as enabled by MDUSS acquisition.

Medical procedures other than EBRT that rely on use of ionizing radiation can benefit from the proposed MDUSS probe. For example, in interventional radiology, catheters are normally guided using x-ray fluoroscopy. MDUSS with remotely located actuators (or radiolucent actuators) could be used to acquire US images during catheter procedures in order to provide real-time volumetric soft-tissue anatomy context while not interfering with periodic x-ray imaging.

Modification of the above-described assemblies and methods for carrying out the invention, combinations between different variations as practicable, and variations of aspects of the invention that are obvious to those of skill in the art are intended to be within the scope of the claims.

What is claimed is:

1. A system for 3D or 4D imaging comprising:
a. an imaging element unit comprising one or more rigidly coupled imaging elements;
b. two or more actuators; and
c. a mechanism coupling the imaging element unit with the two or more actuators to drive the imaging element unit in at least two degrees of freedom in order to acquire 3D or 4D imaging information.

2. The system of claim 1 wherein the one or more imaging elements comprises ultrasound transducer elements.

3. The system of claim 1 wherein the two or more actuators are positioned remotely outside of an irradiating field.

4. The system of claim 1 wherein a first portion of the mechanism extends within an irradiating field and a second portion of the mechanism attached to the two or more actuators extends outside of the irradiating field.

5. The system of claim 1 wherein the imaging element unit and mechanism are configured to result in a nominal imaging artifact when irradiated.

6. The system of claim 1 wherein the two or more actuators are positioned remotely from the imaging element unit.

7. The system of claim 1 wherein the system is configured for diagnostic imaging or radiation therapy.

8. The system of claim 1 wherein the imaging element unit is rotated about a first axis in a first degree of freedom via a first actuator.

9. The system of claim 8 wherein the imaging element unit is rotated about a second axis in a second degree of freedom via a second actuator, wherein the second axis is orthogonal to the first axis.

10. The system of claim 8 wherein the imaging element unit is rotated about a second axis in a second degree of freedom via a second actuator, wherein the second axis is angled relative to the first axis.

11. The system of claim 8 wherein the mechanism coupling the first actuator to the imaging element unit comprises an assembly having a peg non-concentrically coupled to a rotating disk.

12. A method of imaging comprising:
a. providing an imaging element unit comprising one or more rigidly coupled imaging elements, two or more actuators, and a mechanism to couple the imaging element unit with the two or more actuators to drive the imaging element unit in at least two degrees of freedom; and
b scanning a region of a body via the imaging element unit, wherein the imaging element unit is moved relative to the region of the body in a pattern which collectively forms a 3D or 4D image.

13. The method of claim 12 wherein the one or more imaging elements comprise ultrasound transducer elements.

14. The method of claim 12 wherein the two or more actuators are positioned remotely outside of an irradiating field.

15. The method of claim 12 wherein a first portion of the mechanism extends within an irradiating field and a second portion of the mechanism attached to the two or more actuators extends outside of the irradiating field.

16. The method of claim 12 wherein the pattern comprises a series of slices which are substantially planar and respectively formed by rotating the imaging element unit about a first axis in a first degree of freedom.

17. The method of claim 16 wherein the imaging element unit is rotated in a periodic cycle about the first axis to form the respective image slices.

18. The method of claim 16 wherein the series of slices are configured in a substantially radial pattern about a second axis that is contained within the series of slices.

19. The method of claim 16 wherein the series of slices are adjacent and rotated about a second axis that is orthogonal to the first axis.

20. The method of claim 16 wherein the series of slices are adjacent and rotated about a second axis that is angled relative to the first axis.

21. The method of claim 12 wherein the pattern is configured in a spiral trajectory about a central axis.
